(19) United States
(12) Patent Application Publication
(10) Pub. No.: US 2023/0301843 A1
(43) Pub. Date: Sep. 28, 2023

(54) TAMPON/SUPPOSITORY HYBRID

(71) Applicant: Monique Rini, East Brunswick, NJ (US)

(72) Inventor: Monique Rini, East Brunswick, NJ (US)

(21) Appl. No.: 17/705,078

(22) Filed: Mar. 25, 2022

(51) Int. Cl. A61F 13/20; A61F 13/26; A61M 31/00

(52) U.S. Cl. CPC A61F 13/2074; A61F 13/266; A61F 13/2051; A61M 31/007

(57) ABSTRACT

Some implementations can include a tampon/suppository hybrid having at least one composition (e.g., pharmaceutical, medicinal plant, herbal composition, vitamin supplementation, and/or essential oil) applied to, attached to, or contained in the tampon (e.g., at or near an insertion point or tip of the tampon). When the tampon/suppository hybrid is inserted, the tampon/suppository hybrid works to provide the composition to the user, for example, to establish/re-establish the microbial flora of the host, while still absorbing secretions and or blood from menstruation flow.

### FIELD

The present disclosure relates generally to tampons, and, more particularly, to a tampon/suppository hybrid.

### BACKGROUND

Conventional tampons can help provide fluid absorption during menstrual cycles. However, tampon users may benefit from a composition being applied vaginally during menstrual cycles or at other times. Accordingly, a need may exist for a hybrid article that can both act as an absorbent article and suppository to provide one or more compositions during a menstrual cycle of a user.

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

### SUMMARY

Some implementations can include a tampon can include a tampon/suppository hybrid having at least one composition (e.g., pharmaceutical, medicinal plant, herbal composition, vitamin supplementation, probiotic, prebiotic, pharmaceutical, and/or essential oil or the like, or a combination of two or more of the above) applied to, attached to, or contained in an insertion point (or tip), or other part, of the tampon. When the tampon is inserted, the tip of the tampon/suppository hybrid works to provide a compound, for example to establish/re-establish the microbial flora of the host while still absorbing secretions and or blood from menstruation flow.

In some implementations, the tampon can include at least one medicinal plant, herbal composition, vitamin supplementation and or essential oil composition. The composition will be applied or attached to insertion point (tip) of tampon during manufacturing. The composition(s) can also be applied anywhere on the absorbent article, which can be comprised of any cotton, fiber, hemp or material used for absorbency.

The tampon (or absorbent article) can include at least medicinal plant, herbal composition, vitamin supplementation and or essential oil composition at the insertion point (tip) of tampon, acting as a hybrid tampon/suppository which will establish/re-establish the microbial flora of the host during her period. In addition, aiding in restoration and protection of vaginal flora, locally. Vaginal imbalances can cause infections, irritation and unpleasant odor. Some implementations can help to reduce or eliminate some or all of these problems, while maintaining absorbency. There can be many variations of the compositions or proprietary blends used at the insertion point (tip) of the tampon, such as those prone to candida overgrowth, treating bacterial overgrowths or reducing risk of Toxic Shock Syndrome (TSS).

Some implementations can include a tampon/suppository hybrid absorbent article comprising a tampon plunger, a tampon housing, an absorbent insert having a hollowed well at a distal end of the absorbent insert, and a composition disposed in the hollowed well.

In some implementations, the absorbent insert includes one or more of cotton, hemp, bamboo, a synthetic fiber material, or a plant-based fiber material. In some implementations, the composition includes one or more of a medicinal plant composition, an herbal composition, a vitamin or an essential oil composition. In some implementations, the composition includes one or more of a liquid, powder, creme or other encapsulation or packaged composition.

In some implementations, the composition is placed into hollowed well area and adhered to the tampon absorbent insert. In some implementations, the composition is one of freeze dried, molded solid form, or encased in a dissolvable encapsulation.

### BRIEF DESCRIPTION OF THE DRAWINGS

FIG. 1 shows a diagram of an example tampon/suppository hybrid in accordance with some implementations.

### DETAILED DESCRIPTION

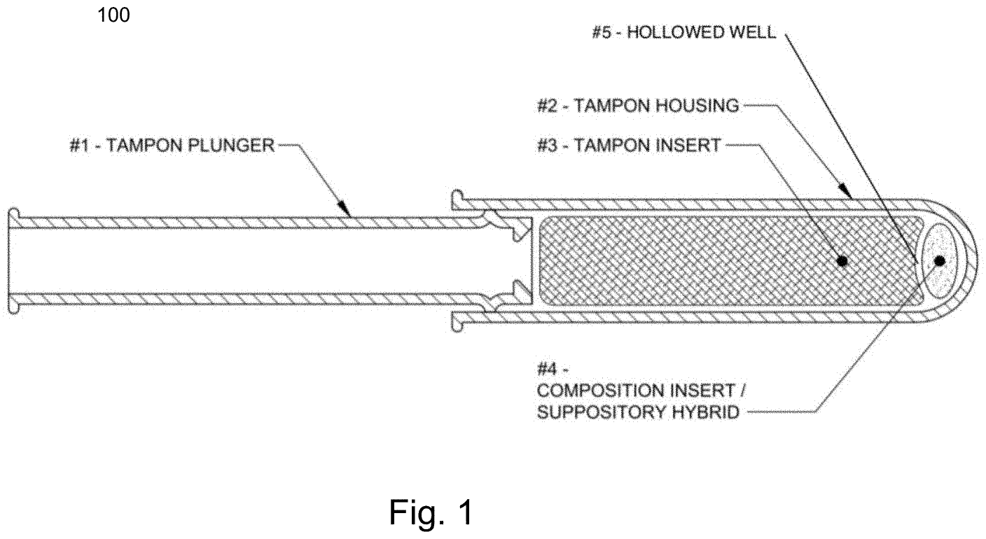

FIG. 1 shows a diagram of an example tampon/suppository hybrid 100 in accordance with some implementations. The tampon/suppository hybrid 100 includes a tampon plunger 1, a tampon housing 2, an absorbent insert 3 (e.g., cotton or other absorbent material), composition 4, and hollowed well 5.

In a manufacturing operation, the hollowed well 5 of the absorbent insert 3 can be filled with a composition 4 including one or more ingredients. The absorbent insert 3 is then placed within the tampon housing 2 along with the plunger 1.

In a use operation, the tampon/suppository hybrid 100 is inserted into the body and the plunger 1 is used to push the absorbent insert 3 (and the composition 4) into the body. The housing 2 and the plunger 1 are then removed from the body.

The tampon/suppository hybrid 100 can be constructed as an absorbent article such as a tampon with a hollowed well 5 at the tip located at insertion point of tampon. The hollowed well 5 can contain a medicinal plant, herbal composition, vitamin and or essential oil composition 4 in the form of a liquid, powder, crème or other encapsulation or packaged composition until flush with outer walls of the absorbent insert 3.

If using a plastic plunger applicator 1, the hollowed well 5 at the tip can hold an encased dissolvable composition 4, encapsulated with at least one medicinal plant, herbal, vitamin supplementation, essential oil compounds and or components, which is then embedded into hollowed well area 5 and fastened or adhered to the tampon absorbent insert 3. The medicinal plant, herbal, vitamin supplementation, essential oil composition can be freeze dried, molded solid form or encased in a dissolvable encapsulation in any shape or size. The microbial ecosystem of a healthy individual can be disturbed by the use of antibiotics, contraceptives, and during hormonal changes such as, menstruation. This can result in a disturbance or imbalance of normal microbial flora which can leave the host susceptible to infections that cause vaginitis, urinary tract infections, candida overgrowth and two most common bacteria found in the diagnosis of toxic shock syndrome (TSS) are Streptococcus pyogenes (group A strep) or Staphylococcus aureus (staph). In some cases of TSS, the strep or staph bacteria may cause further serious infection in the body, such as pneumonia, osteomyelitis, or endocarditis. The vaginal flora includes the bacteria and other organisms that live inside the vagina. The normal vaginal flora are dominated by various lactobacillus species, which help to keep the vagina healthy by producing lactic acid, hydrogen peroxide, and other substances that inhibit the growth of yeast and other unwanted organisms.

The composition can be medicinal including plants, herbs, vitamins and oils or other medicinal or pharmaceutical compositions. Pre/Probiotics can include all bacterium strains that may encourage or promote healthy flora and that may help relieve, reduce or eliminate vaginitis, urinary tract infections, candida overgrowth Streptococcus pyogenes (group A strep), Staphylococcus aureus (staph), Streptococcus B (GBS) as well as other bacterium.

In some implementations, a tampon/suppository hybrid with medicinal properties located at insertion point of (tip) tampon is designed to establish or re-establish the Ph balance of vaginal flora. As the tampon is inserted, the plunger is pushed into the encasement and injects the cotton absorbent article along with the suppository composition into the female genital tract.

There can be many proprietary blends comprised of compounds and/or combination of compounds, medicinal oils, plants and or herbal compositions to target one or more common issues such as candida. There can also be variations of the way the “blends” are applied. Some in freeze dry, molded, encapsulated, soft gel or liquid/cream form. The absorbent article, or tampon can also vary using any material, including but not limited to hemp, natural and/or organic cotton or any other materials used for absorbent tampons. Structural variations of how the variable blends/composition of pre/probiotics, medicinal plant(s), herb(s) or oil(s) can be applied in different locations of absorbent tampon. Location can be at tip and or side (s) of absorbent tampon, including the base or any other location of the absorbent tampon. These methods can also be applied to any absorbent article that is inserted into any orifice of the body. For tampons that do not have an encasing, a thin plastic or aluminum covering can be applied to protect the composition.

It is contemplated that any optional feature of the inventive variations described may be set forth and claimed independently, or in combination with any one or more of the features described herein. Reference to a singular item, includes the possibility that there is a plurality of the same items present. More specifically, as used herein and in the appended claims, the singular forms “a,” “an,” “said,” and “the” include plural referents unless specifically stated otherwise. In other words, use of the articles allow for “at least one” of the subject item.

The term “comprising” in the to be appended claims shall allow for the inclusion of any additional element irrespective of whether a given number of elements are enumerated in the to be appended claim, or the addition of a feature could be regarded as transforming the nature of an element set forth in the claims. Except as specifically defined herein, all technical and scientific terms used herein are to be given as broad a commonly understood meaning as possible while maintaining claim validity.

The breadth of the present disclosure is not to be limited to the examples provided and/or the subject specification, but rather only by the scope of the disclosed subject matter. The disclosed subject matter can include many variations explicitly or implicitly described herein, including those variations that would be obvious to one of ordinary skill in the art upon reading the present specification. Further, it is not intended that any section of this specification (e.g., the Summary, Detailed Description, Abstract, Field, etc.) be accorded special significance in describing the disclosed subject matter relative to another or the claims. All references cited are incorporated by reference in their entirety. Although the foregoing description has been described in detail for purposes of clarity of understanding, it is contemplated that certain modifications may be practiced within the scope of the disclosed subject matter.

What is claimed is:

1. A tampon/suppository hybrid absorbent article comprising:
a tampon plunger;
a tampon housing;
an absorbent insert having a hollowed well at a distal end of the absorbent insert; and
a composition disposed in the hollowed well.

2. The tampon/suppository hybrid absorbent article of claim 1, wherein the absorbent insert includes one or more of cotton or hemp.

3. The tampon/suppository hybrid absorbent article of claim 1, wherein the composition includes one or more of a medicinal plant composition, an herbal composition, a vitamin or an essential oil composition.

4. The tampon/suppository hybrid absorbent article of claim 1, wherein the composition includes one or more of a liquid, powder, creme or other encapsulation or packaged composition.

5. The tampon/suppository hybrid absorbent article of claim 1, wherein the composition is placed into hollowed well area and adhered to the absorbent insert.

6. The tampon/suppository hybrid absorbent article of claim 1, wherein the composition is one of freeze dried, molded solid form, or encased in a dissolvable encapsulation.
